(19) United States
(12) Patent Application Publication
(10) Pub. No.: US 2018/0158549 A1
(43) Pub. Date: Jun. 7, 2018

(54) SURGICAL TRAY COORDINATION SYSTEM AND METHOD

(71) Applicant: Keerthi Kanubaddi, Union, KY (US)

(72) Inventor: Keerthi Kanubaddi, Union, KY (US)

(21) Appl. No.: 15/833,071

(22) Filed: Dec. 6, 2017

Related U.S. Application Data
(60) Provisional application No. 62/430,422, filed on Dec. 6, 2016.

(51) Int. Cl. G16H 40/40; G06Q 10/08; G06Q 30/04

(52) U.S. Cl. CPC G16H 40/40; G06K 19/06028; G06Q 30/04; G06Q 10/087

(57) ABSTRACT

A surgical tray coordination method and system is disclosed that includes receiving and storing tray content information; generating and storing tray tracking information; generating invoices for the trays; recording pick-up of the trays; and tracking the status of each tray from drop-off to pick-up. The surgical tray content information can include a photograph of the tray contents. The tracking information can be on a printed label, which can include descriptive information of tray contents and a tracking code. The method can include sending a notification that a particular tray is checked-in after storing tray content and tracking information. A user can scan the label on any tray to retrieve information regarding the selected tray, including a photograph of tray contents. A user can identify each piece of equipment on a tray; and the system can automatically retrieve a price from a preselected price list; and generate a tray invoice

### CROSS REFERENCE TO RELATED APPLICATIONS

This application claims priority to U.S. Provisional Patent Application Ser. No. 62/430,422, filed Dec. 6, 2016 entitled “Surgical Tray Coordination System;” the disclosure of which is expressly incorporated herein by reference.

### BACKGROUND

The number of people serviced each day at healthcare facilities is increasing at a rapid rate. There are various procedures required to service the incoming patients and various types of specialized equipment is required for each procedure. It is not financially possible or feasible for a hospital or healthcare facility to own and manage all of the surgical equipment and resources that it might use to treat its patients. Hospitals can borrow loaner equipment, consign implants and use other methods to help resolve this issue. However, these methods can introduce new problems, for example poor communication of equipment needs, delays in delivery, delivery of the wrong equipment, missing equipment and strain on human resources to coordinate all of the parties involved.

Hospital efficiency can be compromised and workflow disrupted by poor vendor communication. Surgical delays are estimated to cost the healthcare system billions of dollars. These surgical delays can be attributed to unavailability of the necessary surgical equipment, implant apparatus, loaner equipment and various other reasons. Leveraging electronic devices, for example smart phones, tablets and computers, can improve vendor communications and implant and loaner logistics and reduce delays that add cost to the healthcare system. It would be desirable to be able to use electronic devices to enhance hospital-vendor coordination for greater surgical readiness.

### SUMMARY

A surgical tray coordination method is disclosed that includes receiving and storing surgical tray content information for one or more surgical trays; generating and storing surgical tray tracking information for the one or more surgical trays; generating one or more invoices for equipment on the one or more surgical trays; recording pick-up of the one or more surgical trays; and tracking the status of each of the one or more surgical trays from drop-off through the equipment drop-off module to pick-up through the equipment pick-up module. Receiving and storing surgical tray content information for one or more surgical trays can include storing a photograph of the contents for each of the one or more surgical trays. Generating and storing surgical tray tracking information for the one or more surgical trays can include generating and printing a label for each of the one or more surgical trays with a unique tray identifier. The label for each respective surgical tray of the one or more surgical trays can include descriptive information of the contents of the respective surgical tray and a tracking code for the respective surgical tray.

The surgical tray coordination method can also include sending a notification that a particular surgical tray is checked-in after receiving and storing surgical tray content information for the particular surgical tray, and generating and storing surgical tray tracking information for the particular surgical tray. The surgical tray coordination method can include enabling a user to scan the label on any selected tray of the one or more surgical trays to retrieve information regarding the selected tray, including a photograph of the contents of the selected tray. The surgical tray coordination method can include enabling a user to scan the label on any selected tray of the one or more surgical trays; enabling the user to print a new label to be placed on the selected tray after the selected tray has been sterilized; and generating a notification that the selected tray is ready for use. The surgical tray coordination method can include enabling a user to scan the label on any selected tray of the one or more surgical trays to retrieve information regarding the selected tray, and to generate a usage notice indicating that the selected tray was used.

Generating one or more invoices for equipment on the one or more surgical trays can include enabling a user to identify each piece of equipment on a selected tray of the one or more surgical trays; automatically retrieving a price for each identified piece of equipment from a preselected price list; and generating a tray invoice for the selected tray covering all identified pieces of equipment. The surgical tray coordination method can include enabling a user to approve the tray invoice for the selected tray; and sending a notification that the selected tray has been used.

Recording pick-up of the one or more surgical trays can include enabling a user to scan the label on any selected tray of the one or more surgical trays to retrieve information regarding the selected tray; and enabling the user to accept the selected tray for pick-up.

A surgical tray coordination system is disclosed that includes an equipment drop-off module, an invoice module and an equipment pick-up module wherein the surgical tray coordination system tracks the status of each of the one or more surgical trays from drop-off through the equipment drop-off module to pick-up through the equipment pick-up module. The equipment drop-off module is configured to receive and store surgical tray content information, and store surgical tray tracking information for one or more surgical trays. The invoice module is configured to generate one or more invoices for equipment on the one or more surgical trays. The equipment pick-up module is configured to record pick-up of the one or more surgical trays after use.

The equipment drop-off module can include a photograph tray module configured to store a photograph of the contents for each of the one or more surgical trays; and a label module configured to print a label for each of the one or more surgical trays. The label for each respective surgical tray of the one or more surgical trays includes descriptive information of the contents of the respective surgical tray and a tracking code for the respective surgical tray. After a particular surgical tray of the one or more surgical trays has been dropped-off through the equipment drop-off module, the equipment drop-off module can send a notification that the particular surgical tray is checked-in.

The surgical tray coordination system can include a pre-op module configured to enable a user to scan the label on any selected tray of the one or more surgical trays to retrieve information regarding the selected tray, where the pre-op module enables the user to accept or replace the photograph of the contents for the selected tray, and to print new labels for the selected tray. The surgical tray coordination system can include an operating room (OR) module configured to enable a user to scan the label on any selected tray of the one or more surgical trays to retrieve information regarding the selected tray, and to generate a usage notice indicating that the selected tray was used.

The invoice module can include an invoice generator that enables a user to identify each piece of equipment on a selected tray of the one or more surgical trays, and generate a tray invoice for the selected tray. The invoice generator can automatically retrieve a price for each identified piece of equipment from a preselected price list. The OR module can enable a user to approve the tray invoice for the selected tray; and the surgical tray coordination system can send a notification that the selected tray has been used.

The equipment pick-up module can include a tray review module configured to enable a user to scan the label on any selected tray of the one or more surgical trays to retrieve information regarding the selected tray; and a pick-up accept module to enable the user to accept the selected tray for pick-up.

### BRIEF DESCRIPTION OF THE DRAWINGS

FIG. 1 illustrates an exemplary overview of a vendor representative's interactions with a surgical tray coordination system;

FIG. 2 illustrates an exemplary overview of a hospital personnel's interactions with a surgical tray coordination system;

FIG. 3A illustrates some example pre-op steps for an exemplary surgical coordination procedure;

FIG. 3B illustrates some example operating room and post-op decontamination and sterilization steps for an exemplary surgical coordination procedure;

FIG. 3C illustrates some additional example post-op steps for an exemplary surgical coordination procedure;

FIGS. 4A and 4B illustrate an exemplary flow diagram for tray check-in and pickup procedures in an exemplary surgical coordination system by a vendor representative;

FIGS. 5A and 5B illustrate an exemplary flow diagram for use of an exemplary surgical coordination system by a healthcare user;

FIG. 6 illustrates an example of a log in window;

FIG. 7 illustrates an example of a vendor welcome window;

FIG. 8 illustrates an example of a search window;

FIG. 9 illustrates an example of a case details window;

FIG. 10 illustrates an example of a check-in trays window;

FIG. 11 illustrates an example of a photograph trays window;

FIG. 12 illustrates an example of a printing labels window;

FIG. 13 illustrates an example of a check-in complete window;

FIG. 14 illustrates an example of an invoice window;

FIG. 15 illustrates a general notification message generated by the system;

FIG. 16 illustrates an example of a tray pickup window;

FIG. 17 illustrates an example of a tray label; and

FIG. 18 shows an exemplary hospital computer system with a surgical tray coordination system and exemplary network interfaces and interfacing systems.

### DETAILED DESCRIPTION

The exemplary embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

A surgical procedure requires specific equipment and resources. Some of the equipment and resources may be owned by the hospital and other equipment and resources may be loaned or provided by outside vendors. In addition, a surgical procedure may require resources that are used only in a single procedure, for example surgical implants. A surgical coordination system can be used to help consolidate logistics, confirmation and communication with accessibility by the various members of the surgical team to alleviate many of the issues and inefficiencies involved with coordinating and managing multiple parties in the process.

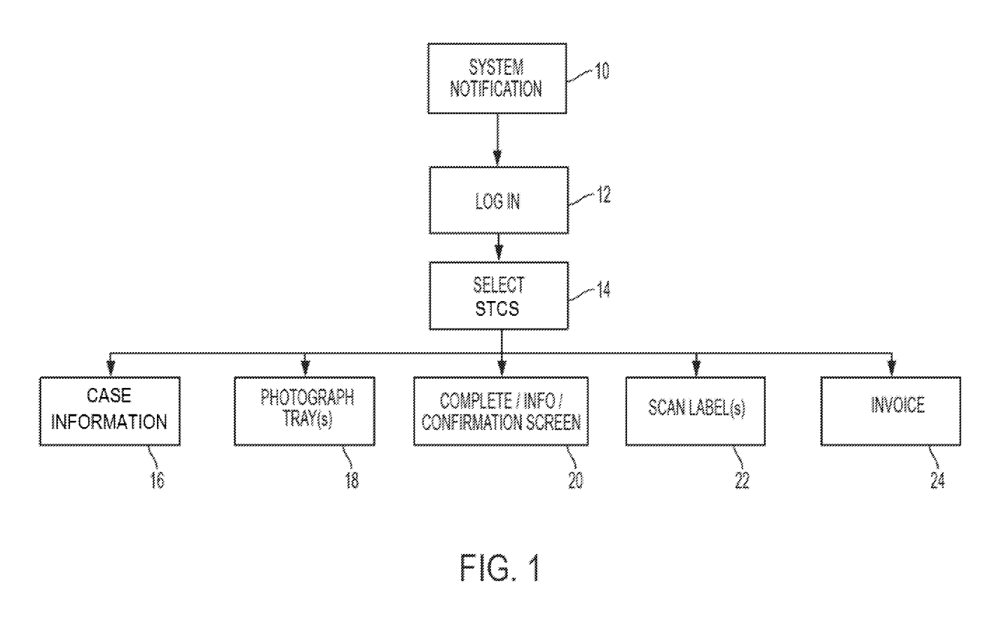

FIG. 1 illustrates an exemplary overview of a vendor representative's interactions with a Surgical Tray Coordination System (STCS). At block 10, the vendor representative receives a notification from the system, for example an invitation to accept a case, a notification that the vendor has accepted a case, a notification of equipment delivery to the hospital, a notification of surgical instrument sterilization, a notification that surgical instruments are ready for pickup, a notification or invitation to replenish surgical instruments or supplies, or other notifications or invitations. The system can automatically send a notification/invitation to the vendor and/or other selected persons when a new surgery or other relevant event is added to the surgical schedule. The notifications/invitations and other correspondence regarding a case can be sent to all or a selected subset of the persons involved with the case. The system can give persons involved with the case the option to opt in or out of notifications regarding the case. At block 12, the vendor representative logs into the surgical tray coordination system. The vendor representative can log into the system using a computer, a smart phone or other electronic device. The vendor representative can log into the system whether or not they received an invitation or notification from the system at block 10. At block 14, after the vendor representative successfully logs into the system, they can select a particular case to which they have access. If the vendor representative received an invitation/notification from the system at block 10, the invitation/notification can include a link that requires the vendor representative to first log into the surgical tray coordination system (block 12) and after successfully logging in automatically takes the vendor representative to the case detail screen for the case associated with the invitation/notification. The case detail for a particular procedure can include the relevant specifications and instructions for that procedure. An invitation/notification for a case can be generated by anyone involved with the case, for example the vendor representative, the surgical scheduler, the surgical team leader, etc. or by a representative appointed by someone involved with the case, for example a hospital or vendor representative.

Either by clicking the link in an invitation/notification or by selecting a case, once logged into the system, the vendor representative can be taken to a desired area regarding the selected case. FIG. 1 shows five exemplary areas but there can be more or less depending on the embodiment of the surgical tray coordination system. Block 16 is a case information area that provides the vendor representative with general information regarding the selected case. Block 18 is a photograph trays area that provides the vendor representative with functionality to photograph trays and store the images in the system. Block 20 is an information entry and confirmation area where the vendor representative can enter and confirm information regarding the selected case. Block 22 is a scan labels area where the vendor representative can scan a tray label, for example by using a smart phone or label scanner, and the system can retrieve images and information regarding the tray associated with the scanned label. Block 24 is an invoice area where the vendor representative can complete, confirm and submit an invoice for the instruments and supplies associated with the tray or trays. The invoice amounts can be tied to a hospital's item master, charge master or vendor contracts or other desired price list. The surgical tray can auto-populate the price information for a surgical tray based on part numbers and item numbers associated with the tray. The system can also transmit this information to be made part of an electronic medical record associated with the case.

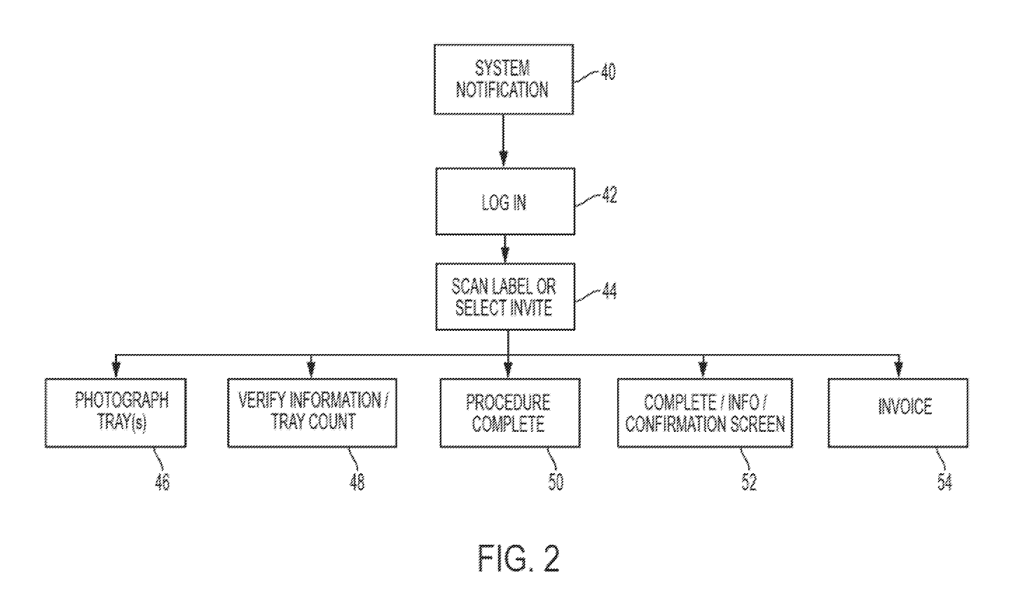

FIG. 2 illustrates an exemplary overview of a hospital user's interactions with a surgical tray coordination system. At block 40, the hospital user receives a notification/invitation from the system. The notifications/invitations and other correspondence regarding a case can be sent to all or a selected subset of the persons involved with the case. The system can give persons involved with the case the option to opt in or out of notifications regarding the case. At block 42, the hospital user logs into the surgical tray coordination system. The hospital user can log into the system using a computer, a smart phone or other electronic device. The hospital user can log into the system whether or not they received an invitation or notification from the system at block 40. At block 44, after the hospital user successfully logs into the system, they can select a particular case to which they have access. If the hospital user received an invite/notification from the system at block 40, the invitation/notification can include a link that requires the hospital user to first log into the surgical tray coordination system (block 42) and after successfully logging in automatically takes the hospital user to the case detail screen for the case associated with the invitation/notification. Alternatively after successfully logging in, the hospital user can scan a surgical tray label and the system can automatically take the hospital user to the case detail screen for the case associated with the tray label. The case detail for a particular procedure can include the relevant specifications and instructions for that procedure.

Once logged into the system, the hospital user can be taken to a desired area regarding the selected case. FIG. 2 shows five exemplary areas but there can be more or less depending on the embodiment of the surgical tray coordination system. Block 46 is a photograph trays area that provides the hospital user with functionality to photograph trays and store the images in the system. Block 48 is a case information/tray count area that provides the hospital user with case information and tray information for the selected case. Block 50 is a procedure complete area where the hospital user can indicate that the selected case is complete. Block 52 is an information entry and confirmation area where the hospital user can enter and confirm information regarding the selected case. Block 54 is an invoice area where the hospital user can review, update and verify an invoice for the instruments and supplies associated with the tray or trays. The hospital user can receive a notification that a procedure is complete with a link that automatically takes them to block 50, 52 and/or 54 to verify that the procedure is complete and to review and verify information regarding the procedure.

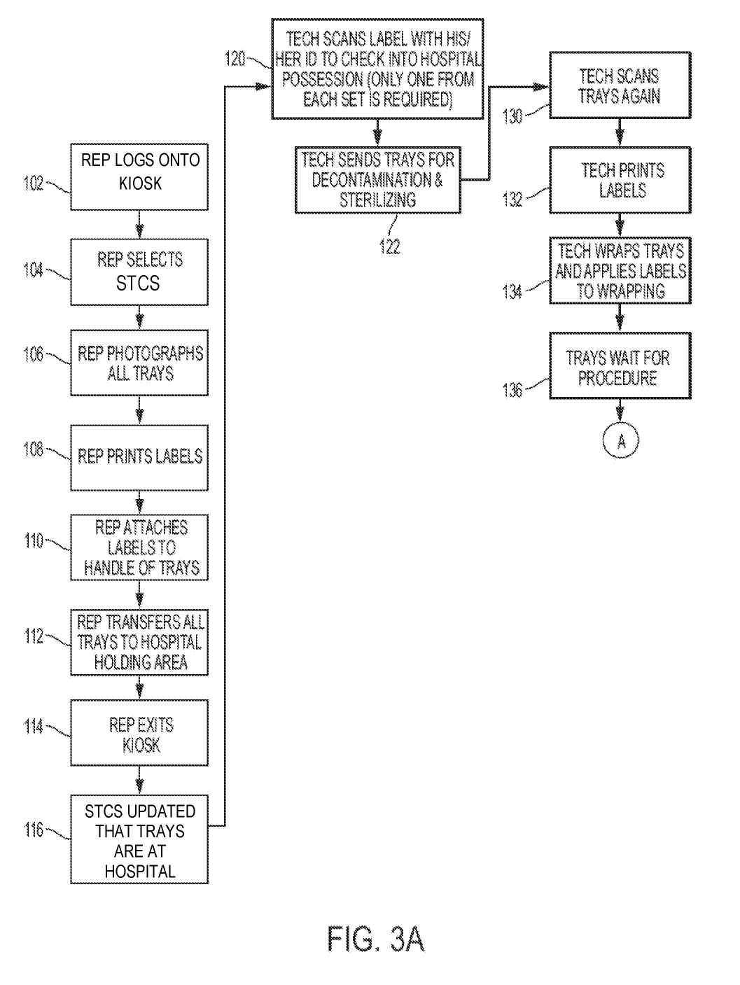

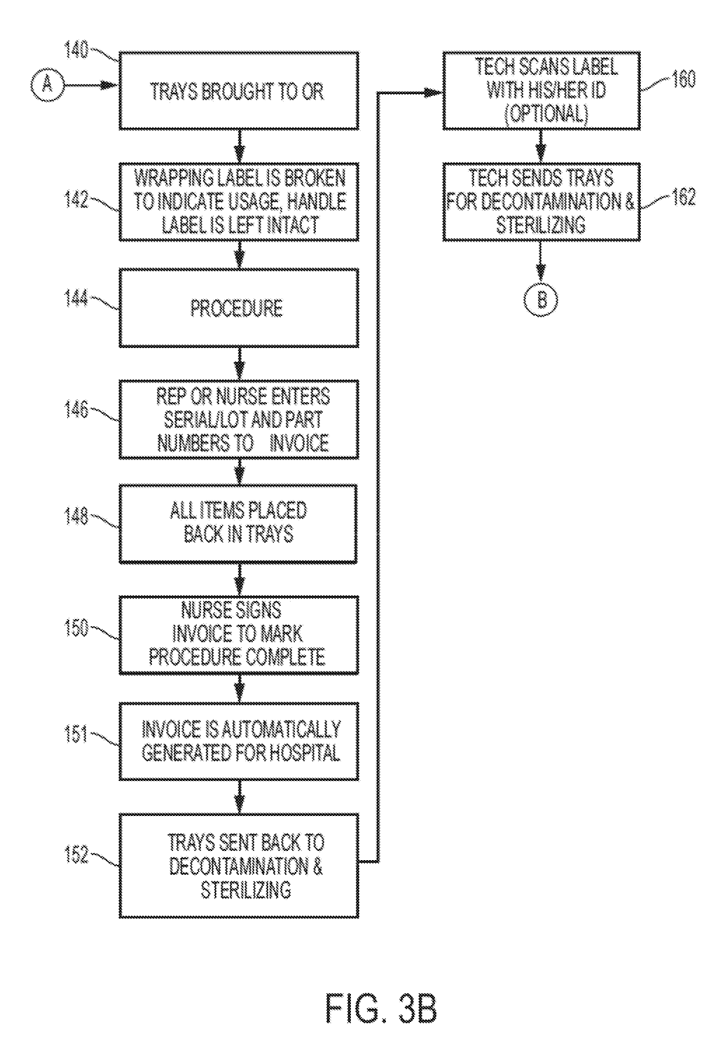

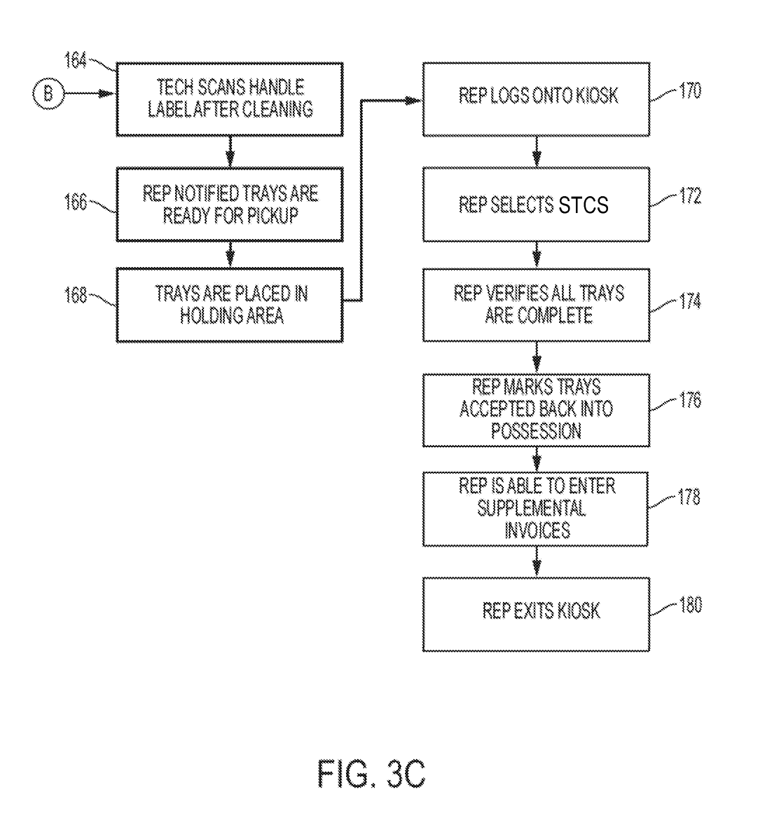

The surgical coordination system can be used to log delivery of surgical equipment and/or resources by vendors, to track sterile processing and preparation for the surgery, to track equipment delivery and sterilization, to track surgery uses and returns, and other aspects of surgical procedures. FIGS. 3A, 3B and 3C show steps that can be part of an exemplary surgical procedure 100.

FIG. 3A shows some example pre-op steps for the exemplary surgical procedure 100. At block 102, a vendor representative logs onto the surgical coordination system. At block 104, the vendor representative selects a module of the surgical coordination system to enter equipment delivered for a surgical procedure. In this example, the vendor delivers surgical trays containing equipment to be used in the upcoming surgical procedure. At block 106, the vendor representative photographs all of the surgical trays showing all of the surgical equipment delivered and these photographs are stored in the surgical coordination system. At block 108 the vendor representative uses the surgical coordination system to print a label for each of the surgical trays delivered, and at block 110 the vendor representative attaches the appropriate label to each of the surgical trays delivered. This label printed and attached by the vendor representative can be a handle label attached to the handle of a surgical tray. At block 112 the vendor representative transfers all of the delivered surgical trays to a hospital holding area, and at block 114 the vendor representative logs out of the surgical coordination system. At block 116, the surgical coordination system updates its surgical database to indicate that the surgical trays delivered by the vendor representative are now at the hospital.

At block 120, a technician or other healthcare staff member scans his or her identification badge and then scans the labels of the surgical trays delivered by the vendor representative to check the surgical trays into hospital possession. At block 122, the healthcare staff member sends the surgical trays to decontamination and sterilization for surgical preparation.

At block 130, a technician or other healthcare staff member in the decontamination and sterilization group (D/S staff member) scans his or her identification badge and scans the labels of the trays to confirm delivery of the trays to the decontamination and sterilization department. For each surgical tray, the D/S staff member sterilizes all of the equipment on the tray; at block 132 prints a new label for the sterilized tray; and at block 134 wraps the sterilized tray and applies the new label to the wrapping. At block 136, the surgical coordination system updates its surgical database to indicate that the surgical trays are now sterilized and ready for the surgical procedure.

FIG. 3B shows some example operating room (OR) and post-op decontamination and sterilization steps for the exemplary surgical procedure 100. At block 140, the sterilized surgical tray is brought into the OR by an OR staff member. The sterilization label can be scanned by an OR staff member, and if so, the surgical coordination system can update the database to indicate that the surgical tray is being or has been used in the OR. At block 142, the wrapping label is broken and the handle label is left intact. At block 144, the surgical procedure takes place. At block 146, a vendor representative or hospital staff member uses the surgical coordination system to enter the serial/lot and part numbers for the equipment on the surgical tray onto a single invoice. The equipment that the vendor representative or hospital staff member logs into the surgical coordination system can reside on one or more trays, but the vendor representative or hospital staff member will want to capture what was billed or used on that patient procedure. The invoice generated by the surgical coordination system can be tied to the original contract between the hospital and the vendor, the hospital charge-master or the hospital item-master.

At block 148, the OR personnel puts the equipment back on the surgical tray. At block 150, the surgical nurse signs the invoice to mark the surgical procedure is complete. At block 152, the surgical coordination system automatically generates the surgical tray invoice for the hospital and sends the invoice information to the electronic medical record as a log of implants and disposables used. The surgical coordination system can also automatically send the invoice to the purchasing department for Purchase Order generation, and send a notification to the vendor that X number of items were used and they need to be replenished to the surgical tray from which those items originated. At block 154, the surgical tray is sent back to the D/S group for decontamination and sterilization.

At block 160, a D/S staff member scans his or her identification badge and scans the labels of the trays to confirm delivery of the trays from the OR to the decontamination and sterilization department. At block 162, the D/S staff member sterilizes all of the equipment on the tray. In some instances the trays do not go through a decontamination and sterilization procedure.

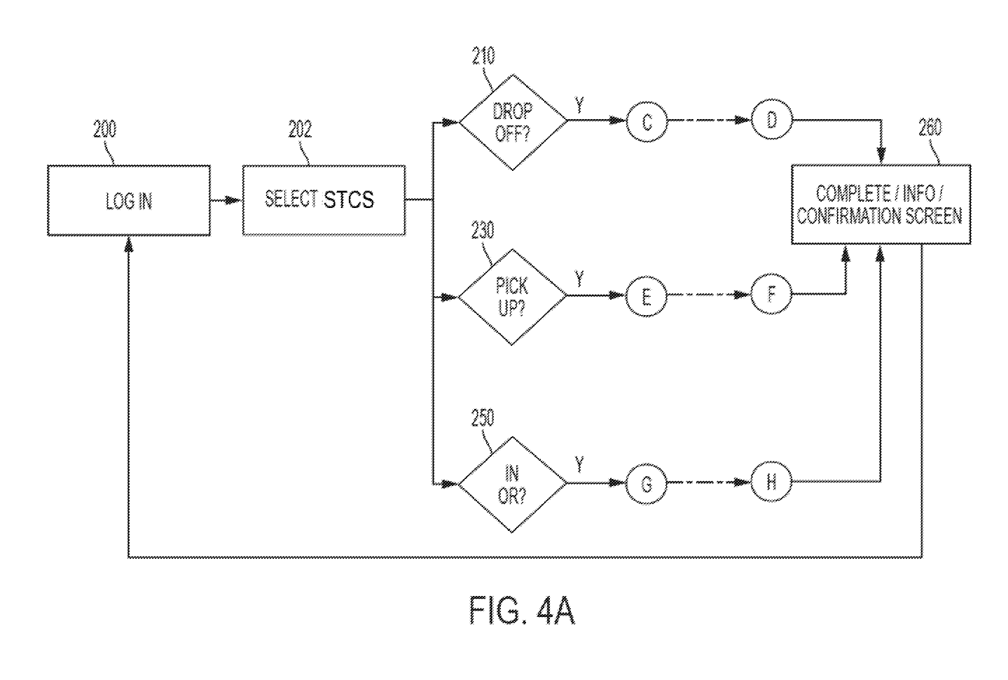

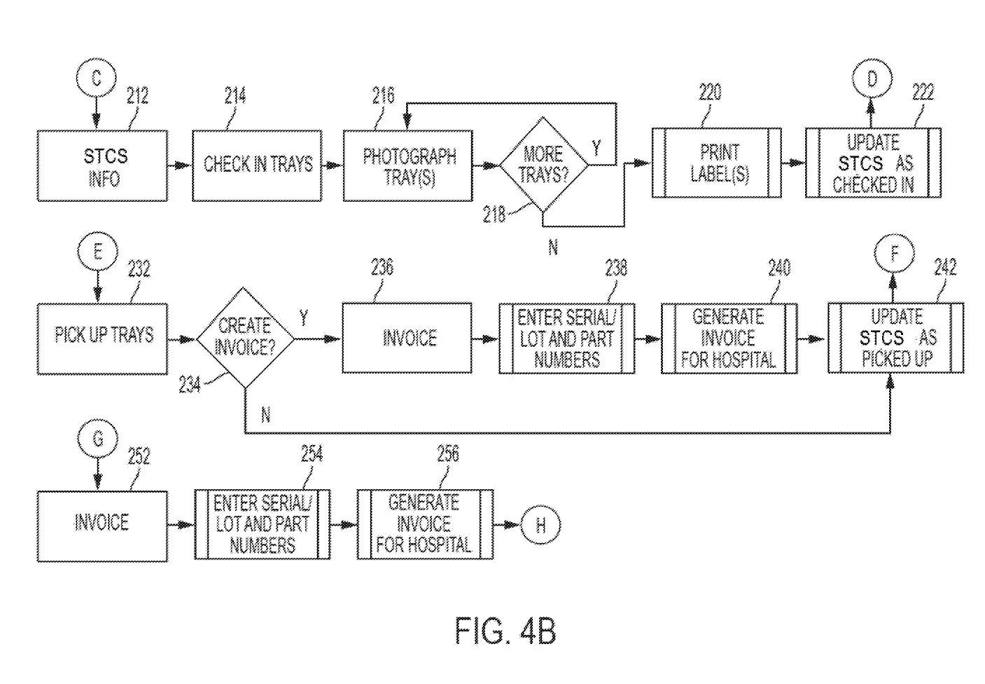

FIG. 3C shows some additional example post-op steps for the exemplary surgical procedure 100. At block 164, the D/S staff member scans the handle label on the tray after cleaning. At block 166, the surgical coordination system updates its surgical database to indicate that the surgical tray has been cleaned after the surgical procedure and notifies the vendor representative that the surgical tray is ready for pickup. At block 168, the cleaned surgical tray is placed in the hospital holding area for vendor pickup. For trays that do not go through a D/S procedure, the system can notify the vendor representative that the trays are ready for pickup a preselected time after surgery. The system can continue to send reminders to the vendor representative that the trays are ready for pickup until the action is complete. Blocks 170-180 provide a brief flow of an exemplary vendor pickup procedure, and will be referenced below with regard to FIGS. 4A and 4B.

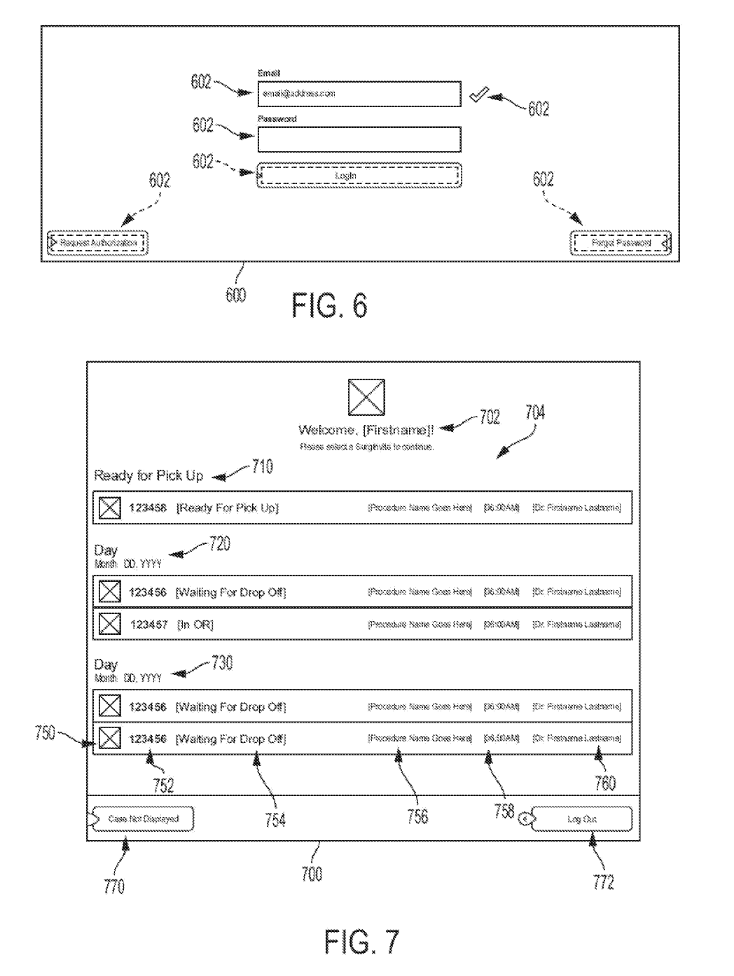

FIGS. 4A and 4B show an exemplary flow diagram for vendor check-in and pickup procedures in a surgical coordination system. At step 200, the vendor representative logs into the surgical coordination system (also shown in block 170 of FIG. 3C). The vendor representative can access the surgical coordination system by various methods, for example by using a kiosk located at the hospital, or by using their smart phone or tablet device while logged into the hospital network. FIG. 6 shows an example log in window 600 that includes a user identifier field 602, a password field 604 and a LOG IN button 606. The LOG IN button 606 may be disabled until the user enters a user identifier in the user identifier field 602 and a password in the password field 604. When the LOG IN button 606 is selected, the surgical coordination system checks that the user entered a valid user identifier in the user identifier field 602 and the associated password in the password field 604; and if so the surgical coordination system allows the vendor representative to access the system and records the actions of the vendor representative. The log in window 600 can place a checkmark 608 next to each field once the field receives a valid input, for example, a properly formatted email address in the user identifier field 602. Other user identifiers besides email addresses can be used by the surgical coordination system. The log in window 600 can also include a Request Authorization button 610 and a Forgot Password button 612. The Request Authorization button 610 can be selected to open a new window that enables a user to request authorization to access the surgical coordination system. The Forgot Password button 612 can be selected to enable the user to reset their password through a secure process.

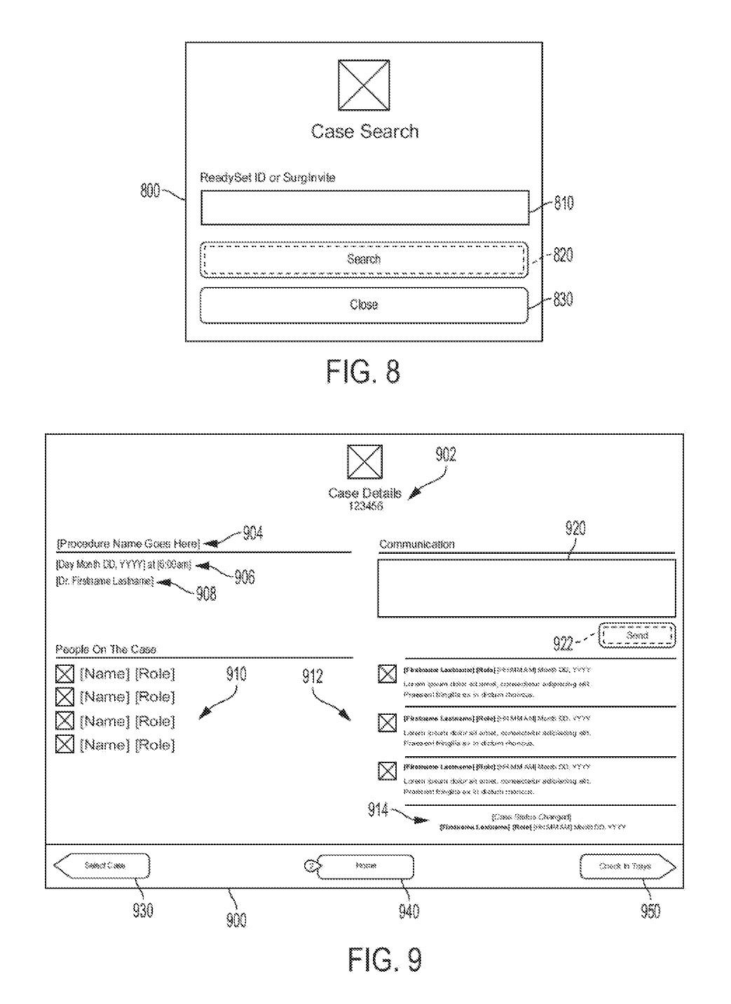

When the vendor representative successfully logs onto the surgical coordination system, they are taken to step 202 (also block 172 of FIG. 3C) where a vendor welcome screen is shown. An example of a vendor welcome screen 700 is shown in FIG. 7. The exemplary vendor welcome screen 700 includes a vendor representative identity field 702 and a listing of applicable procedures 704 for the vendor representative. In this example, the listing of applicable procedures 704 is separated into a Ready for Pick Up group 710 of completed procedures, and pending or active procedure groups 720, 730 grouped by scheduled day. The Ready for Pick Up group 710 includes completed procedures that have vendor equipment ready for pickup or in preparation for vendor pick up. The pending or active procedure groups 720, 730 show procedure and equipment delivery status for each procedure scheduled at the healthcare location that involves equipment from the vendor. The user can select any displayed procedure to interact with the surgical coordination system for that procedure. Each row includes a status icon 750, a procedure identifier 752, a status descriptor 754, a procedure name 756, a scheduled procedure time 758, and a doctor name 760. The example vendor welcome screen 700 also includes a case not displayed button 770 and a log out button 772. If a desired procedure is not displayed in the listing of applicable procedures 704, the vendor can select the case not displayed button 770 to display a search window where the vendor can manually search for a desired procedure by procedure number or other appropriate search criteria. An example search window 800 is described below with reference to FIG. 8. The vendor can select the log out button 772 to sign out of the surgical coordination system and be taken back to the login screen.

FIG. 8 illustrates an exemplary search window 800 when the vendor selects the case not displayed button 770. The search window 800 includes a search field 810, a search button 820 and a close button 830. The user can enter search criteria into the search field 810. In the example of FIG. 8, a procedure identifier 752 is entered into the search field 810, but other search criteria can be used in different embodiments of a surgical coordination system. When the user selects the search button 820, the surgical coordination system performs a search for any cases or procedures that include the search criteria in the search field 810. If one or more cases are found, the search window 800 is closed and the user is taken to a listing of cases meeting the search criteria similar to the listing of procedures 704 shown in FIG. 7. If no case is found, an error message is displayed in the search window 800. If no search criteria is entered in the search field 810, then a listing of all cases for the vendor are displayed in a listing of cases similar to the listing of procedures 704 shown in FIG. 7. When the user selects the close button 830, the surgical coordination system closes search window 800 without performing further searching.

When the user selects a case or procedure from the listing found in a search or from the listing of procedures 704 shown in FIG. 7, the surgical coordination system displays details for the selected case. FIG. 9 shows an example of a case details window 900 that includes a procedure identifier 902, a procedure name 904, a scheduled procedure time 906, and a doctor name 908 which should match the information in the listing found in a search or from the listing of procedures 704. The exemplary case details window 900 also shows a listing of other people on the case 910, a message log for the case 912, and a cases status change entry 914 of who last changed the case status and when the last change was made. The messaging system automatically creates a channel for communication among all the users involved in a case. System generated messages are automatically posted on the messaging platform and user generated messages can also be sent. If a user generated message is marked critical (critical box checked) that message is also sent via Email and SMS to all the users involved in the case depending on their ability to receive these types of messages. This messaging platform ensures all case related communication are stored in one place, for example the message log 912, which can enhance the speed of decision making by those involved in the case.

The system can automatically send out reminder messages and notifications to the appropriate users based on a set of business rules. For example, the system can automatically generate and send a message to the appropriate users for any upcoming cases at 48 hours before surgery time, for any upcoming cases at 24 hours before surgery time, upon any case acceptance by a vendor or other user, when trays are to be delivered to the hospital for a case, when trays are to be collected from the hospital after surgery, etc. Based on these rules, notification messages are generated automatically by the system and distributed using any available modes, for example Email, SMS, etc.

The exemplary case details window 900 also includes a message field 920 and a send button 922. The user can enter a message to be added to the communications in the surgical communication system for the selected procedure into the message field 920. When the user has finished typing the message in the message field 920, the user can select the send button 922 to send the message. When a message is created within a case detail, the message can go to all users assigned to that case. If the message is selected as “High Priority”, the message can be sent to the inbox of all Users as a “High Priority” message and be treated and displayed accordingly. When the send button 922 is selected, the message is also added to the communications history in the surgical communication system for the selected procedure.

The exemplary case details window 900 also includes a back button 930, a home button 940 and a next button 950. When the back button 930 is selected, the surgical communication system goes back to the previous screen. The back button 930 can show the screen name of the previous screen to provide context to the user. In this exemplary case details window 900, the previous screen is a vendor welcome screen 700. When the home button 940 is selected, the surgical communication system goes back to the vendor welcome screen 700. When the next button 950 is selected, the surgical communication system goes to the next screen in the process. The next button 950 can show the screen name of the next screen to provide context to the user. In this exemplary case details window 900, the next screen is a check-in trays screen 1000. The next button 950 can be disabled until the user completes any necessary information on the current screen.

Going back to FIG. 4A and the exemplary vendor welcome screen 700, each procedure in the listing of applicable procedures 704 for the vendor representative has a status descriptor 754. In this example, the status descriptor 754 is either “Ready for Pick Up”, “In OR”, or “Waiting for Drop Off”. From block 202, if the vendor representative selects a procedure from the listing of applicable procedures 704 with a status descriptor 754 of “Waiting for Drop Off”, then control passes through block 210 to the equipment drop-off process starting at block 212 shown in FIG. 4B. If the vendor representative selects a procedure from the listing of applicable procedures 704 with a status descriptor 754 of “Ready for Pick Up”, then control passes through block 230 to the equipment pickup process starting at block 232 shown in FIG. 4B. If the vendor representative selects a procedure from the listing of applicable procedures 704 with a status descriptor 754 of “In OR”, then control passes through block 250 to the OR invoice process starting at block 252 shown in FIG. 4B.

The equipment drop-off process starting at block 212 is reached when the vendor representative selects a selected procedure from an applicable procedures list 704 or a procedures list from a search with a status descriptor 754 of “Waiting for Drop Off.” At block 212, a case details window 900 for the selected procedure is shown and the next button 950 can show “check-in trays” as the screen name of the next screen. The user can review the information on the case details window 900 and can send a message for the case using the message field 920 and send button 922. When the user selects the next button 950, the surgical communication system proceeds to block 214 where a check-in trays window is displayed.

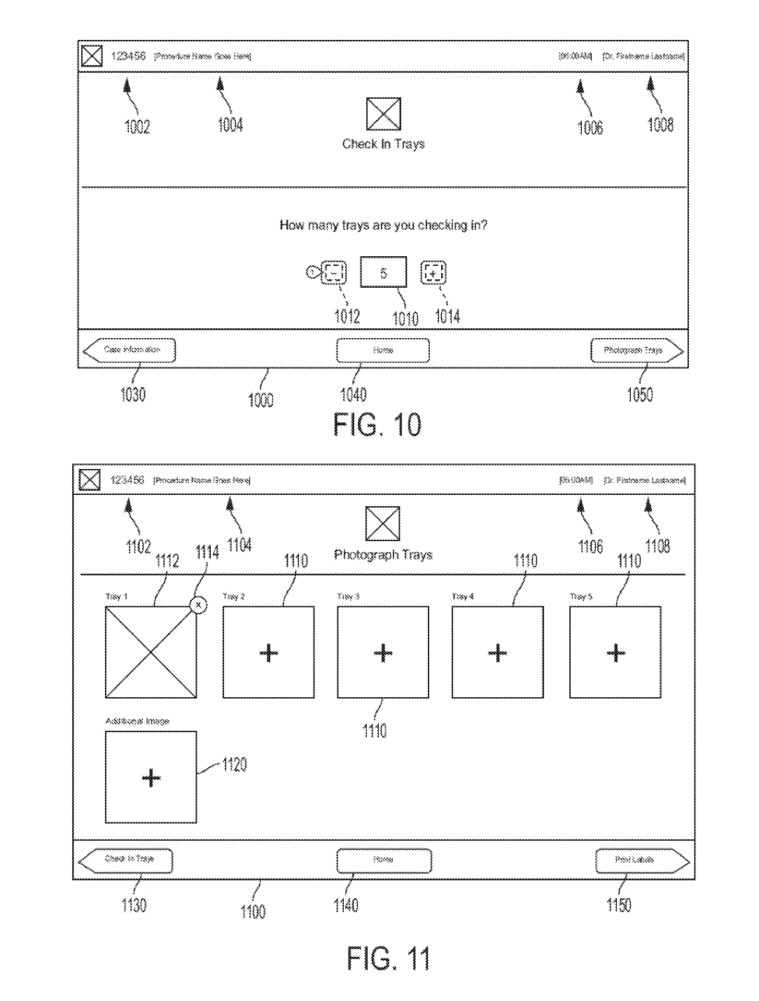

FIG. 10 shows an example of a check-in trays window 1000. The exemplary check-in trays window 1000 includes a procedure identifier 1002, a procedure name 1004, a scheduled procedure time 1006, and a doctor name 1008 for the selected procedure. The exemplary check-in trays window 1000 includes a tray count field 1010, a tray increment button 1012 and a tray decrement button 1014. The tray increment button 1012 increments the number in the tray count field 1010, and the tray decrement button 1014 decrements the number in the tray count field 1010. The user uses the tray increment and decrement buttons 1012, 1014 to change the number in the tray count field 1010 until it shows the correct number of trays being checked in for the selected procedure.

The exemplary check-in trays window 1000 also includes a back button 1030, a home button 1040 and a next button 1050. When the back button 1030 is selected, the surgical communication system goes back to the previous screen. The back button 1030 can show the screen name of the previous screen to provide context to the user. In this exemplary check-in trays window 1000, the previous screen is a case details window 900 for the selected case. When the home button 1040 is selected, the surgical communication system goes back to the vendor welcome screen 700. When the next button 1050 is selected, the surgical communication system goes to the next screen in the process. The next button 1050 can show the screen name of the next screen to provide context to the user. In this exemplary check-in trays window 1000, the next screen is a photograph trays window 1100. The next button 1050 can be disabled until the user enters a valid number in the tray count field 1010. When the user selects the next button 1050, the surgical communication system proceeds to block 216 where a photograph trays window is displayed.

FIG. 11 shows an example of a photograph trays window 1100. The exemplary photograph trays window 1100 includes a procedure identifier 1102, a procedure name 1104, a scheduled procedure time 1106, and a doctor name 1108 for the selected procedure. The exemplary photograph trays window 1100 includes add image buttons 1110 for the number of trays selected in the tray count field 1010 of the check-in trays window 1000. When the vendor checks in one or more trays, the kiosk can enable the vendor to take a picture of each tray and its contents and print the associated sticker. The user can select one of the add image buttons 1110 and add an image for the specified tray. When the user adds an image, the add image button 1110 is replaced with a tray image 1112 just taken or added by the user and a delete button 1114 appears for each tray image 1112. FIG. 11 shows the tray image 1112 already added for Tray 1, and add image buttons 1110 for Trays 2-5. The user can select the delete button 1114 to remove the associated tray image 1112. The exemplary photograph trays window 1100 includes an additional image button 1120 to enable the user to add additional images beyond the specified number of tray images. When the additional image button 1120 is selected, an additional image added by the user appears in the former location of the additional image button 1120, and the additional image button 1120 duplicates to the right of the newly added image to allow the user to continue adding as many images to the case as desired. Each tray can have multiple images because a single tray can be designed to have multiple levels.

The exemplary photograph trays window 1100 also includes a back button 1130, a home button 1140 and a next button 1150. When the back button 1130 is selected, the surgical communication system goes back to the previous screen. The back button 1130 can show the screen name of the previous screen to provide context to the user. In this exemplary photograph trays window 1100, the previous screen is a check-in trays window 1000 for the selected case. When the home button 1140 is selected, the surgical communication system goes back to the vendor welcome screen 700. When the next button 1150 is selected, the surgical communication system goes to the next screen in the process. The next button 1150 can show the screen name of the next screen to provide context to the user. In this exemplary photograph trays window 1100, the next screen is a print labels window 1200. The next button 1150 can be disabled until the user enters a photograph for each tray.

At block 216 of FIG. 4B, when a tray image 1112 is added by the user to replace an add image button 1110, the system proceeds to block 218 to check if there are more add image button(s) 1110 to be replaced by tray images. If more tray images are needed, then the system returns to block 216 for the tray images to be added. If no more tray images are needed, then the system can proceed to block 220 to print labels for the delivered trays. The next button 1150 can be disabled until the user enters a photograph for each tray. When the user selects the next button 1150, the surgical communication system proceeds to block 220.

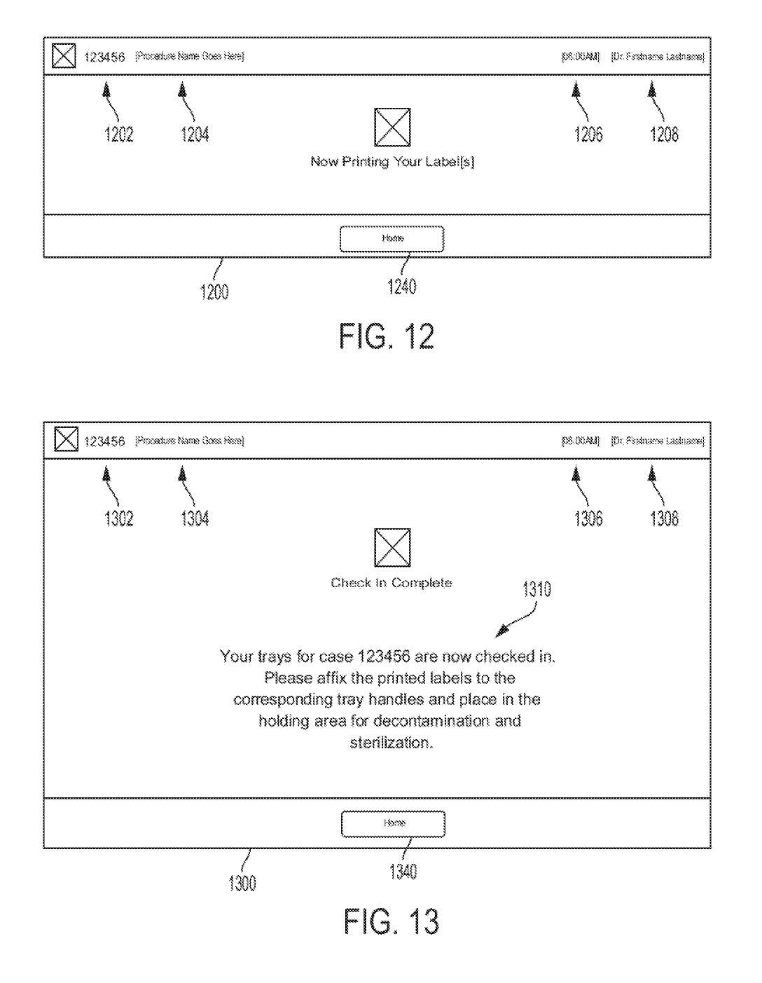

At block 220, the surgical communication system prints a label for each of the delivered trays that identifies the tray is intended for the selected case or procedure. FIG. 12 shows an example of a printing labels window 1200. The exemplary printing labels window 1200 includes a procedure identifier 1202, a procedure name 1204, a scheduled procedure time 1206, and a doctor name 1208 for the selected procedure. The exemplary printing labels window 1200 also includes a home button 1240 to allow the user to look at other procedures or cases while the tray labels are being printed. When the home button 1240 is selected, the surgical communication system goes back to the vendor welcome screen 700.

FIG. 17 shows an example of a tray label 1700. The exemplary tray label 1700 includes a Quick Response (QR) code 1702, a procedure identification field 1704, a tray content description 1706, a multi-tray indicator 1708, a tray preparation description 1710, and a barcode 1712. The Quick Response (QR) code 1702 enables users to scan the code and quickly view the contents and case details associated with the particular surgical instrument tray or implant. The QR code 1702 can provide a type of x-ray vision for the tray, in that when the QR code 1702 is scanned it can show the contents of the tray without having to open the tray. The procedure identification field 1704 can include the procedure date, time and doctor name for the selected procedure. The tray content description 1706 provides a brief description of the contents of the tray. The multi-tray indicator 1708 indicates whether the tray content is divided across multiple trays and the order of the surgical trays containing the tray content. The tray preparation description field 1710 can provide information regarding the manufacture and preparation of the tray contents. The barcode 1712 can correspond to a barcode from an alternative tracking system within the hospital so that the surgical tracking system can interact with legacy and other hospital systems. The linear barcode 1702 can be used to scan trays into various other hospital systems.

When all the tray labels are printed, the surgical communication system proceeds from block 220 to block 222 where the equipment drop-off and check-in process is completed. At block 222, the status of the selected procedure is updated; for example, the status descriptor 754 is changed from “Waiting for Drop Off” to “Checked-In.” A check-in complete window can also be displayed. FIG. 13 shows an exemplary check-in complete window 1300. The exemplary check-in complete window 1300 includes a procedure identifier 1302, a procedure name 1304, a scheduled procedure time 1306, and a doctor name 1308 for the selected procedure. The exemplary check-in complete window 1300 also includes instructions 1310 for the vendor to affix the printed tray labels to the delivered trays and place the labeled trays in the holding area. The exemplary check-in complete window 1300 also includes a home button 1340 to allow the user to go back to the vendor welcome screen 700. From block 222 the surgical communication system proceeds to block 260 shown in FIG. 4A.

The equipment pickup process starting at block 232 is reached when the vendor representative selects a selected procedure from an applicable procedures list 704 or a procedures list from a search with a status descriptor 754 of “Ready for Pick Up.” At block 232, a case details window 900 for the selected procedure is shown and the next button 950 can show “Invoice” or “Pick-Up” as the screen name of the next screen depending on whether or not an invoice has already been created for the use of this equipment. The user can invoice at this point for missing equipment, but they would usually submit the procedural invoice when the tray is in the OR with the surgical team as the products are being used. The user can review the information on the case details window 900 and can send a message for the case using the message field 920 and send button 922. From block 232, the system proceeds to block 234.

At block 234, the user selects whether they want to create an invoice for the use of the equipment in the procedure. This is also shown at block 178 of FIG. 3C. If an invoice is to be created, the system proceeds to block 236. If an invoice is not to be created, the system proceeds to block 242.

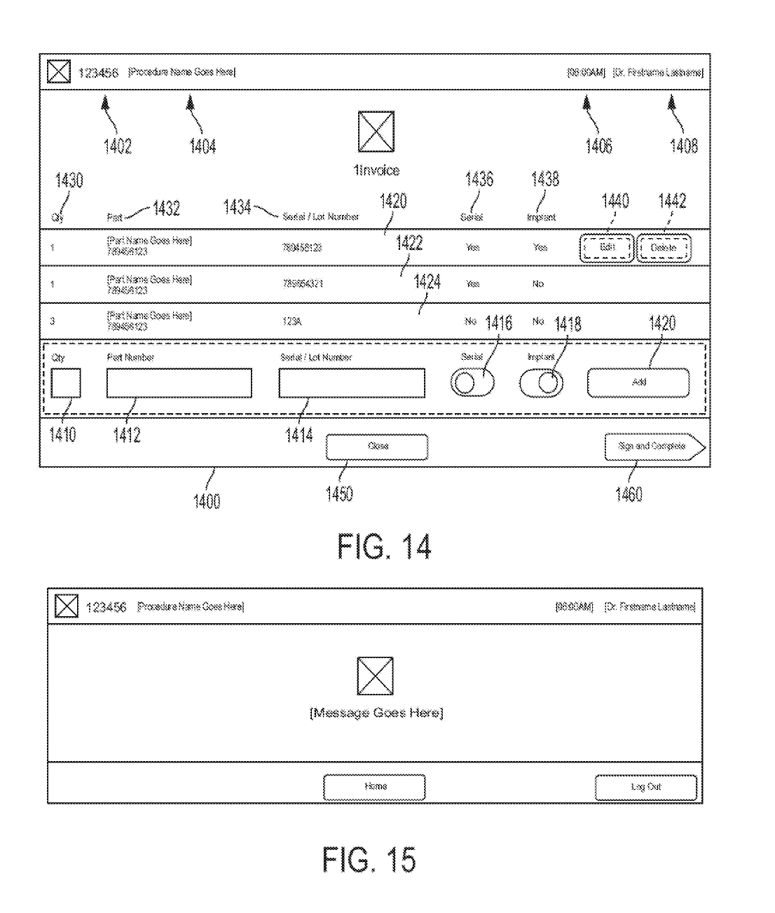

At block 236, the surgical communication system starts the invoice creation process and displays an invoice screen for generating an invoice for the equipment used in the procedure. The invoice screen is also used for block 236 to enter serial/lot and part numbers and block 238 to generate an invoice for the hospital. FIG. 14 shows an example of an invoice screen 1400.

The exemplary invoice screen 1400 includes a procedure identifier 1402, a procedure name 1404, a scheduled procedure time 1406, and a doctor name 1408 for the selected procedure. The invoice screen can include invoice row entry fields and one or more invoice rows. The exemplary invoice screen 1400 shows the following invoice row entry fields: a quantity entry field 1410, a part number entry field 1412, a serial number entry field 1414, a serial number toggle 1416, and an implant toggle 1418. The serial number toggle 1416 can indicate whether the product has a serial number or not, since some medical equipment only has a part number and lot number and not a serial number. The implants toggle 1418 can identify if the item used was an implant. The user enters the appropriate information in the input fields 1410, 1412, 1414 and puts the toggles 1416, 1418 in the desired positions. When the input entry fields 1410-1418 have the correct information, the user selects an add button 1420 and a new invoice row is created with the information from the invoice row entry fields. The invoice row entry fields can be cleared when the add button 1420 is selected to prepare for entry of the next invoice row values. Invoice rows can be automatically saved as they are added to the invoice so if the user closes and returns to the invoice, all previously entered and undeleted invoice rows are retained.

The exemplary invoice screen 1400 has three invoice rows 1420, 1422, 1424. Each invoice row includes a quantity entry 1430, a part number entry 1432, a serial/lot number entry 1434, a serial number indicator 1436, and an implant value 1438. When the serial number indicator 1436 is “Yes” it indicates that the serial/lot number entry 1434 is a serial number, and when the serial number indicator 1436 is “No” it indicates that the serial/lot number entry 1434 is a lot number. When an invoice row is selected, such as invoice row 1410, additional functionality including an edit button 1440 and a delete button 1442 are displayed. Selecting the edit button 1440 enables the user to edit the entries and values in the selected invoice row. Selecting the delete button 1442 enables the user to delete the selected invoice row.

The exemplary invoice screen 1400 also includes a close button 1450 and a complete and sign button 1460. Selecting the close button 1450 closes the invoice window 1400. Selecting the complete and sign button 1460 finishes the invoice process by allowing the OR nurse to sign the screen to accept the invoice. The vendor usually generates the invoice when they are present in the procedure, however there can be instances when the hospital staff will need to create the invoice. The sign-off screen is usually used by the circulating nurse.

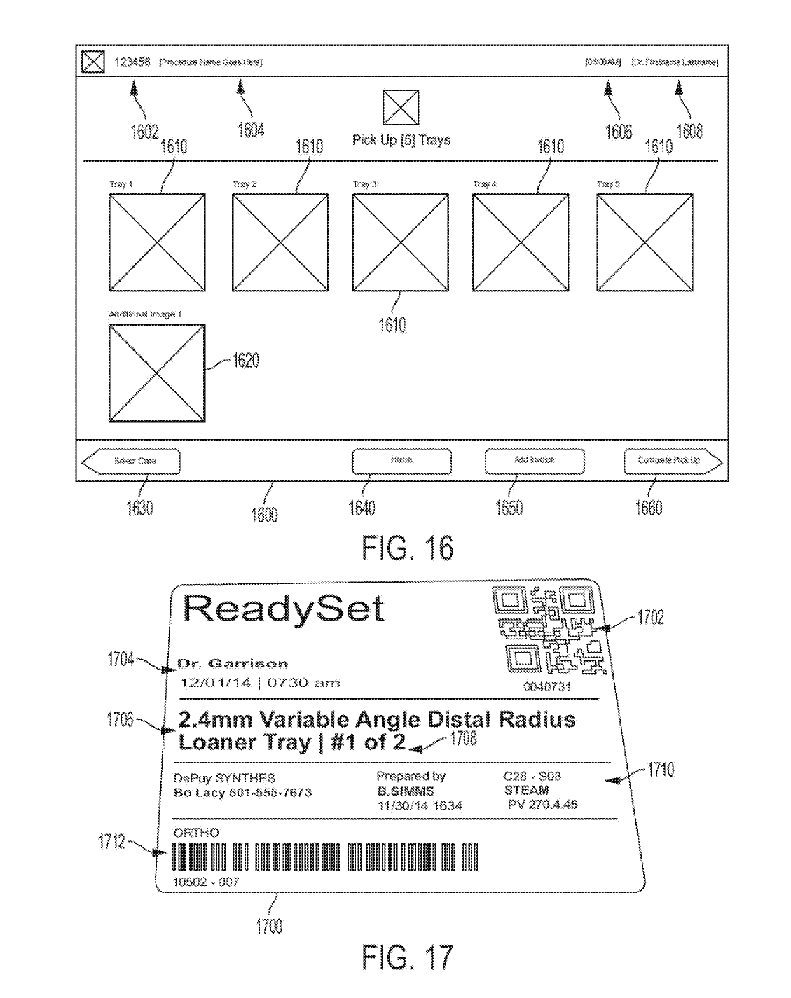

The pickup trays process proceeds to block 242 after the invoice process at block 240 or if no invoice is created at block 234. At block 242 a tray pickup window is displayed. FIG. 16 shows an example of a tray pickup window 1600.

The exemplary tray pickup window 1600 includes a procedure identifier 1602, a procedure name 1604, a scheduled procedure time 1606, and a doctor name 1608 for the selected procedure. The exemplary tray pickup window 1600 includes tray image(s) 1610 and additional image(s) 1620 that were submitted by the user in the photograph trays window 1100 when checking in the trays. These images can be used to verify the tray contents when picking up the trays. The exemplary tray pickup window 1600 also includes a back button 1630, a home button 1640, and add invoice button 1650 and a complete pickup button 1660. When the back button 1630 is selected, the surgical communication system goes back to the previous screen. The back button 1630 can show the screen name of the previous screen to provide context to the user. In this exemplary tray pickup window 1600, the previous screen is a case details window 900 where the vendor can select the case from which they are picking up equipment. When the home button 1640 is selected, the surgical communication system goes back to the vendor welcome screen 700. When the add invoice button 1650 is selected the user is taken through the invoice process as explained above with reference to FIG. 14 and blocks 236-240. The user can use the invoice process to submit a new or additional invoice to the case. When the vendor representative verifies that the trays are complete (block 174 of FIG. 3C), then they can mark the trays accepted (block 176 of FIG. 3C) by selecting the complete pickup button 1660. When the complete pickup button 1660 is selected, the surgical communication system completes the tray pickup process and the status of the selected procedure is updated; for example, the status descriptor 754 is changed from “Ready for Pick Up” to “Picked Up.” Any supplemental or additional invoice created at block 178 or step 240 can also be submitted as part of completion of the tray pickup process. A pickup complete window can also be displayed and the vendor representative can exit the tray pickup process (block 180 of FIG. 3C). From block 242 the surgical communication system proceeds to block 260 shown in FIG. 4A.

The OR invoice process starting at block 252 is reached when the vendor representative selects a selected procedure from an applicable procedures list 704 or a procedures list from a search with a status descriptor 754 of “In OR.” At block 252, a case details window 900 for the selected procedure is shown and the next button 950 can show “Invoice” as the screen name of the next screen. The user can review the information on the case details window 900 and can send a message for the case using the message field 920 and send button 922. In blocks 252, 254, 256 the surgical communication system performs the invoice creation process using the invoice screen for generating an invoice for the equipment used in the procedure. The invoice creation process is described above with reference to FIG. 14 and blocks 236-240. From block 256 the surgical communication system proceeds to block 260 shown in FIG. 4A.

At block 260, the system can return to the case selection screen 700 where the user can select the log out button.

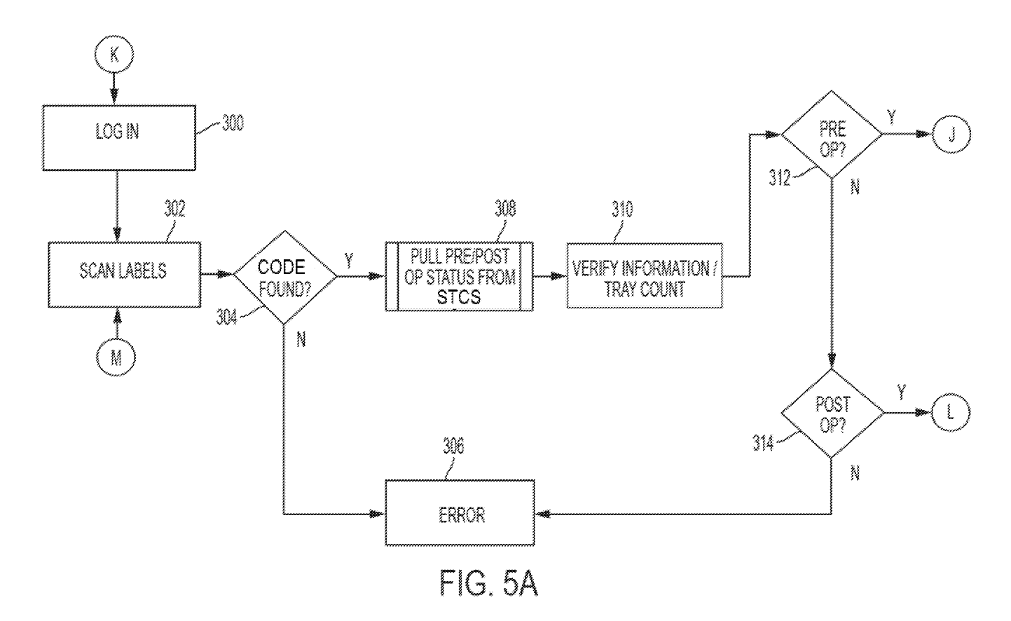

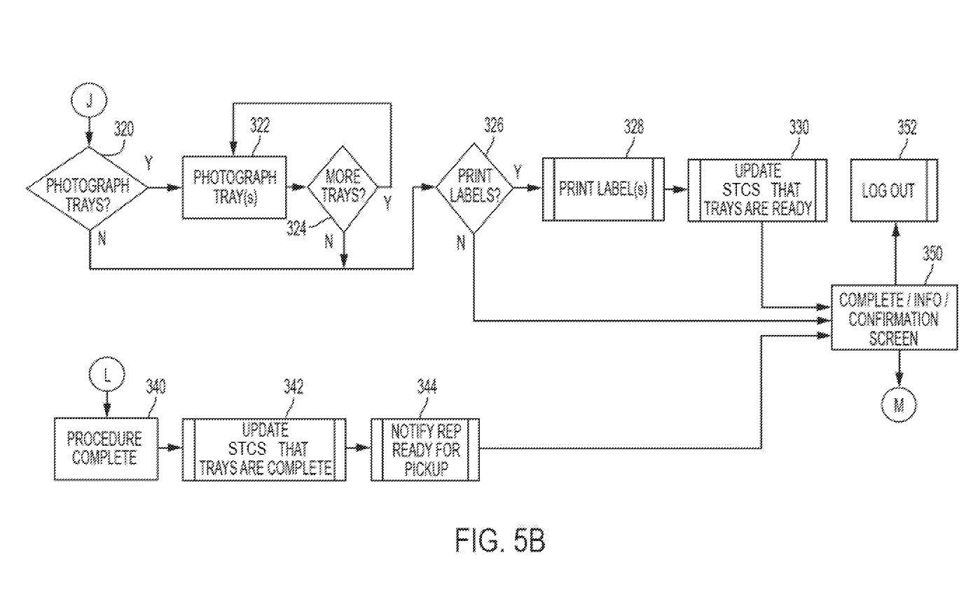

FIGS. 5A and 5B show an exemplary flow diagram for healthcare users use of the surgical coordination system. At block 300, the healthcare user logs in to the surgical coordination system. The log in window and functionality can be the same as described above with reference to block 200 of FIG. 4A, and FIG. 6 for the vendor representative.

When the healthcare user successfully logs on to the surgical coordination system, they are taken to step 302 where they can scan a tray label. When a tray label is scanned the system proceeds to block 304 where it attempts to find the tray label in its database. If the scanned tray label is not found, then at block 306 an error message is displayed. The error procedure can allow the healthcare user to retry the scan or to select a tray by another method. If the scanned tray label is found in the database, then control passes to block 308.

At block 308, the surgical coordination system pulls the Pre/Post operation status for the scanned tray and at block 310 displays the tray information. This can be displayed using the case details window 900, an example of which is shown in FIG. 9.

From block 310, if the tray is pre-procedure then control passes through block 312 to block 320 in FIG. 5B. From block 310, if the tray is post-procedure then control passes through block 312 to block 314 to block 340 in FIG. 5B. From block 310, if the tray is neither pre-procedure nor post-procedure then control passes through block 312 to block 314 to block 306 and an error message is displayed. The error message can return the user to the case details window 900.

At block 320, a window showing the photographs of the trays taken by the vendor is displayed and the user is given the choice of accepting the vendor photographs or taking new photographs. For example, a display similar to one shown in FIG. 11 or FIG. 16 can be used to show the photographs of the trays. If the user decides to take new photographs of the trays, then the system proceeds to block 322 where a photograph trays window is displayed. If the user decides to accept the vendor photographs of the trays then the system proceeds to block 326.

At block 322, a photograph trays window is displayed. An example of a photograph trays window 1100 is shown in FIG. 11. If the user decides to take new photographs of the trays then the vendor photos can be treated as “Additional Photos”. The user can replace all of the vendor photos, or they can accept some and replace others. When a replacement photo is taken, then at block 324 the system checks if there are more trays. If there are more trays then control goes back to block 322 to take photographs of the additional trays. If there are not more trays then control proceeds to block 326.

At block 326, the user is given an option to print labels. If the user decides not to print new labels, then the system proceeds to block 350. If the user decides to print new labels, then at block 328 new labels are printed, and at block 330 the procedure status is updated. The system proceeds to block 350.

At block 350 the user is taken to an information completion and confirmation area where they can enter information regarding the selected case and confirm existing information regarding the case. From block 350, the user can go to block 352 to log out of the system or go to block 302 to scan labels. The user may want to go to block 302 to scan one or more tray labels associated with the procedure to confirm the contents of the tray or trays used in the procedure.

At block 340, a procedure complete process is performed. The procedure complete process can include a user indicating to the system that the procedure is complete, or the system can automatically generate a procedure complete verification message a certain time period after the procedure is scheduled to start, for example 24 hours after procedure start. The procedure complete verification message can be sent to a hospital representative to verify that the procedure has been completed. After it is verified that the procedure is complete, the procedure status can be updated. Updating the procedure status to complete can include, at block 342 confirming that the trays used in the procedure are complete, and at block 344 generating a notification to the vendor representative to pick-up and/or replenish surgical instruments and/or supplies. FIG. 15 shows a generic notification screen that can be sent to the vendor representative and this screen can include a link that automatically takes the vendor representative to the appropriate screen after successfully logging into the system. Alternatively, the vendor representative can receive a procedure complete notification automatically generated by the system, and then the vendor representative can perform block 342 to verify that the trays are complete and pick up the used trays or resubmit some or all of the contents of the surgical tray for use in another procedure.

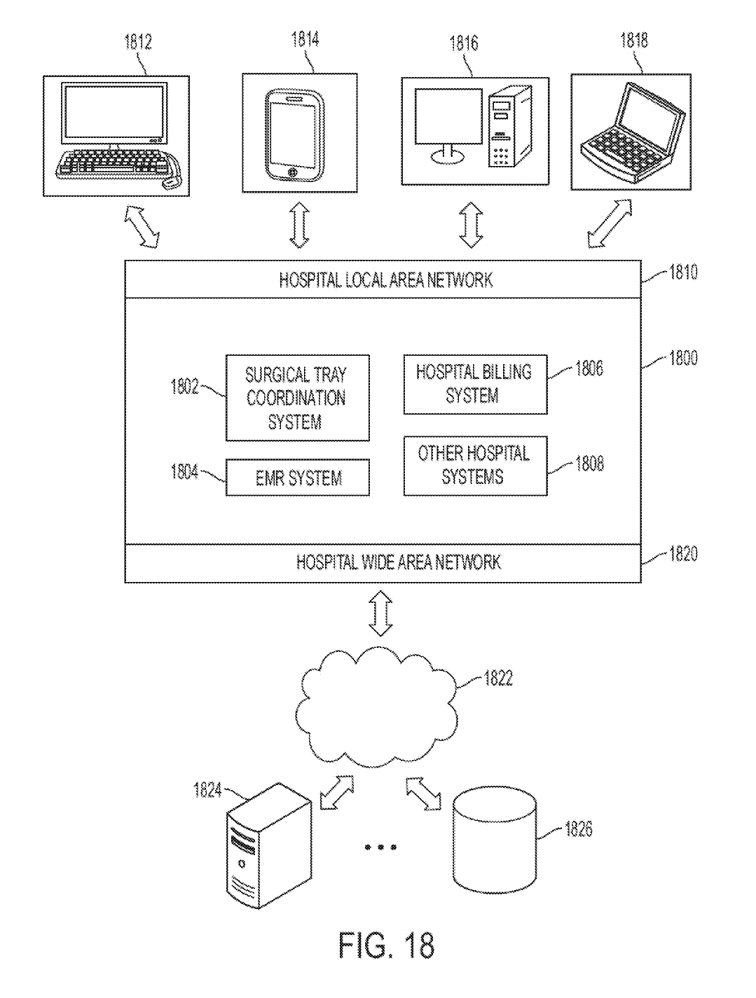

FIG. 18 shows an exemplary hospital computer system 1800 with a surgical tray coordination system 1802 and exemplary network interfaces and interfacing systems. The hospital computer system 1800 includes the surgical tray coordination system 1802, an electronic medical record system 1804, a hospital billing system 1806 and other hospital systems 1808. The hospital computer system 1800 also includes a local area network interface 1810 and a wide area network interface 1820. These various systems 1802-1808 can interface and exchange data with one another directly and through various network interfaces. The local area network interface 1810 can be connected to one or more terminals 1812, smart phones or tablets 1814, desktop computers 1816, laptop computers 1818 and various other electronic devices that can have integrated or connected peripherals such as cameras, QR code readers, barcode readers and other devices used by the system. The wide area network interface 1820 can be connected to the Internet, cloud or other networks 1822 that are connected to various computers, servers and computer systems 1824 and databases 1826. The computers, servers and databases 1824, 1826 can include vendor systems and hospital systems.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains.

We claim:

1. A surgical tray coordination method comprising:
receiving and storing surgical tray content information for one or more surgical trays;
generating and storing surgical tray tracking information for the one or more surgical trays;
generating one or more invoices for equipment on the one or more surgical trays;
recording pick-up of the one or more surgical trays; and
tracking the status of each of the one or more surgical trays from drop-off through the equipment drop-off module to pick-up through the equipment pick-up module.

2. The surgical tray coordination method of claim 1, wherein receiving and storing surgical tray content information for one or more surgical trays comprises storing a photograph of the contents for each of the one or more surgical trays.

3. The surgical tray coordination method of claim 2, wherein generating and storing surgical tray tracking information for the one or more surgical trays comprises generating and printing a label for each of the one or more surgical trays with a unique tray identifier.

4. The surgical tray coordination method of claim 3, wherein the label for each respective surgical tray of the one or more surgical trays includes descriptive information of the contents of the respective surgical tray and a tracking code for the respective surgical tray.

5. The surgical tray coordination method of claim 3, further comprising:
sending a notification that a particular surgical tray is checked-in after receiving and storing surgical tray content information for the particular surgical tray, and generating and storing surgical tray tracking information for the particular surgical tray.

6. The surgical tray coordination method of claim 3, further comprising:
enabling a user to scan the label on any selected tray of the one or more surgical trays to retrieve information regarding the selected tray, including a photograph of the contents of the selected tray.

7. The surgical tray coordination method of claim 6, further comprising:
enabling a user to scan the label on any selected tray of the one or more surgical trays;
enabling the user to print a new label to be placed on the selected tray after the selected tray has been sterilized; and
generating a notification that the selected tray is ready for use.

8. The surgical tray coordination method of claim 7, further comprising:
enabling a user to scan the label on any selected tray of the one or more surgical trays to retrieve information regarding the selected tray, and to generate a usage notice indicating that the selected tray was used.

9. The surgical tray coordination method of claim 8, wherein generating one or more invoices for equipment on the one or more surgical trays comprises:
enabling a user to identify each piece of equipment on a selected tray of the one or more surgical trays;
automatically retrieving a price for each identified piece of equipment from a preselected price list; and
generating a tray invoice for the selected tray covering all identified pieces of equipment.

10. The surgical tray coordination method of claim 9, further comprising:
enabling a user to approve the tray invoice for the selected tray; and
sending a notification that the selected tray has been used.

11. The surgical tray coordination method of claim 10, wherein recording pick-up of the one or more surgical trays comprises:
enabling a user to scan the label on any selected tray of the one or more surgical trays to retrieve information regarding the selected tray; and
enabling the user to accept the selected tray for pick-up.

12. A surgical tray coordination system comprising:
an equipment drop-off module configured to receive and store surgical tray content information, and store surgical tray tracking information for one or more surgical trays;
an invoice module configured to generate one or more invoices for equipment on the one or more surgical trays;
an equipment pick-up module configured to record pick-up of the one or more surgical trays;
wherein the surgical tray coordination system tracks the status of each of the one or more surgical trays from drop-off through the equipment drop-off module to pick-up through the equipment pick-up module.

13. The surgical tray coordination system of claim 12, wherein the equipment drop-off module comprises:
a photograph tray module configured to store a photograph of the contents for each of the one or more surgical trays; and
a label module configured to print a label for each of the one or more surgical trays.

14. The surgical tray coordination system of claim 13, wherein the label for each respective surgical tray of the one or more surgical trays includes descriptive information of the contents of the respective surgical tray and a tracking code for the respective surgical tray.

15. The surgical tray coordination system of claim 13, wherein after a particular surgical tray of the one or more surgical trays has been dropped-off through the equipment drop-off module, the equipment drop-off module sends a notification that the particular surgical tray is checked-in.

16. The surgical tray coordination system of claim 13, further comprising:
a pre-op module configured to enable a user to scan the label on any selected tray of the one or more surgical trays to retrieve information regarding the selected tray;
wherein the pre-op module enables the user to accept or replace the photograph of the contents for the selected tray, and to print new labels for the selected tray.

17. The surgical tray coordination system of claim 16, further comprising:
an operating room (OR) module configured to enable a user to scan the label on any selected tray of the one or more surgical trays to retrieve information regarding the selected tray, and to generate a usage notice indicating that the selected tray was used.

18. The surgical tray coordination system of claim 17, wherein the invoice module comprises:
an invoice generator that enables a user to identify each piece of equipment on a selected tray of the one or more surgical trays, and generate a tray invoice for the selected tray;
wherein the invoice generator automatically retrieves a price for each identified piece of equipment from a preselected price list.

19. The surgical tray coordination system of claim 18, wherein the OR module further enables a user to approve the tray invoice for the selected tray; and
the surgical tray coordination system sends a notification that the selected tray has been used.

20. The surgical tray coordination system of claim 19, wherein the equipment pick-up module comprises:
a tray review module configured to enable a user to scan the label on any selected tray of the one or more surgical trays to retrieve information regarding the selected tray; and
a pick-up accept module to enable the user to accept the selected tray for pick-up.
